(19) United States
(12) Patent Application Publication
(10) Pub. No.: US 2009/0024058 A1
(43) Pub. Date: Jan. 22, 2009

(54) Device for Sampling Oral Fluid

(75) Inventors: Raphael Blowick, Galway (IE); Attracta Roach, Berwick (AU); David Smart, Moira (IE); Sarah Noonan, Newport (IE)

Correspondence Address: FOLEY HOAG, LLP;PATENT GROUP (w/ISA), 155 SEAPORT BLVD., BOSTON, MA, 02210-2600, US

(73) Assignee: Inverness Medical Switzerland GmbH, Zug (CH)

(21) Appl. No.: 11/909,586

(22) PCT Filed: Mar. 20, 2006
(86) PCT No.: PCT/GB2006/000994

§ 371 Date: Aug. 18, 2008

(30) Foreign Application Priority Data
Mar. 22, 2005 (GB) 0505828.4

(51) Int. Cl. A61B 5/00; B01J 19/00; G01N 30/02; C12M 1/34

(52) U.S. Cl. 600/582; 422/68.1; 422/70; 435/287.1

(57) ABSTRACT

A device for the collection of oral fluid from a subject's oral cavity may include a sample-collecting mouthpiece and a one-way valve. The mouthpiece may include a collecting chamber, introducible into the oral cavity of the subject for collecting oral fluid, and a resiliently deformable wall section defining at least part of the collecting chamber such that compression of the resiliently deformable wall forcibly expels a collected fluid from the collecting chamber via a collecting chamber outlet. The one-way valve may be in fluid communication with the collecting chamber outlet and may allow fluid to be displaced from the device following compression of the resiliently deformable wall but prevents ambient air flowing back through the valve into the device.

### FIELD OF THE INVENTION

The present invention relates to a device and method for the collection of an oral fluid from a subject.

### BACKGROUND OF THE INVENTION

The detection of various analytes in tissues and fluids of subjects is of great importance in the diagnosis of infection and disease. At present, most diagnostic testing is carried out using samples of blood, urine, fecal material or tissue biopsy. However, these methods of testing usually involve complicated procedures and may, particularly in the case of blood testing, present a significant safety hazard. In contrast, the testing of oral fluids such as saliva and mucosal transudate is a much simpler process and is relatively safe.

The possible use of oral fluids as clinical specimens for the diagnosis of disease states has been under investigation for some time. Evidence has shown that oral fluids may provide useful samples for the detection of analytes, due to the fact that analytes present in the blood pass through the oral mucosa and/or salivary glands into the oral cavity where they can subsequently be detected. In addition, it is thought that the concentration of analyte in the oral fluid is indicative of the concentration of analyte present in the blood. Thus, much research is being carried out in order to develop devices for the collection of such oral fluids, and to develop assay systems for the presence of analytes in oral fluid.

However, in contrast with diagnostic tests performed with blood, urine or fecal samples where a high volume of sample is available for assay, the amount of oral fluid obtained is often small. Thus, a problem exists in obtaining a sample of oral fluid of sufficiently large volume to permit the performance of one or more assays.

One infection whose detection is of particular importance is that caused by the organism Group A β-hemolytic streptococcus. Of the several groups of Streptococci, group A Streptococcus (S. pyrogenes) is primarily responsible for the morbidity associated with a number of pathological conditions in humans, such as β-hemolytic pneumonia, scarlet fever, rheumatic fever, septic sore throat and cardiac sequelae. Due to the serious nature of infections that may be caused by Streptococcus A (hereinafter referred to as Strep A), it is of extreme importance to diagnose its presence early in infection, such that an appropriate course of treatment may be selected and commenced without undue delay.

Conventional methods of testing for Strep A involve the rubbing of a swab over a patient's throat in order to obtain a sample containing bacterial cells, adding an extraction reagent (such as nitrous acid) to the swab sample in order to dissolve the bacterial cell wall and release the Streptococcus antigens, preparing a fluid sample and assaying the sample for the presence of either Strep A antigen or antibodies to the antigen, using, for example, a conventional lateral flow test-strip. Such processes are discussed in EP No. 280557 (Eastman Kodak Company) and U.S. Pat. No. 4,673,639 (Slifkin). However, these processes often involve several stages and are therefore frequently long and laborious.

Thus, detection of the presence of Strep A in oral fluids such as saliva would confer a significant advantage over these prior art methods, avoiding the requirement for an extraction stage and thus minimising the number of steps involved. In general, devices that have been developed for the collection of oral fluids can be divided into three classes. The first class includes devices that have an absorbent material for absorption of the oral fluid. These devices have as a common feature an absorbent material which is placed in the mouth of the subject and absorbs the oral fluid. The absorbent material is subsequently removed from the mouth of the subject and the oral fluid is subsequently extracted therefrom.

However, the requirement for an absorbing material in these devices limits the range of substances that may be analysed, due to the fact that the absorptive material may irreversibly trap some analytes. Thus, liquid extractant reagents have been used to assist in the removal of absorbed analytes. The use of such liquid extractants may result in the additional problems of sample dilution and non-reproducibility of results, thus reducing the accuracy and reliability of such absorbent collection devices when quantitative analysis is required. One such method of using an absorbent material to collect the sample is disclosed in WO 02/063297 (Avitar, Inc.), wherein a foam member absorbs the fluid sample and subsequently delivers the fluid sample from the foam member onto a test membrane. In addition, U.S. Pat. No. 6,416,715 (Gambert et al) discloses a device which comprises a porous unit that can absorb the sample fluid which can then be squeezed out and collected. However, such methods may result in the non-reversible absorption of some analytes. In addition, lengthy collection times and sample dilution result in a decreased accuracy and precision in quantitative analysis.

The second class of devices includes those which involve the use of osmotic absorption in order to obtain the sample. An example of such a device is disclosed in U.S. Pat. No. 4,817,632 (Schramm) which refers to a device for the collection of oral fluid wherein the device comprises a semi-permeable membrane which defines an enclosed chamber. The semi-permeable membrane allows the passage of the desired oral fluid molecules, but prevents the passage of larger particles or molecules present in the oral fluid. Thus, the device creates an osmotic pressure for drawing the oral fluid from the oral cavity of a subject into the enclosed chamber of the semi-permeable membrane. The sample is subsequently extracted from the chamber with a syringe. However, the use of such devices requires the presence of trained personnel or laboratory equipment in order to analyse the oral fluids and obtain results.

The third class includes devices wherein oral fluids are collected by aspiration methods. Such methods are disclosed in U.S. Pat. No. 6,022,326 (Tatum) and WO 00/25666 (Tatum). These disclose the use of a vacuum (preferably limited to 50-200 Torr) and the second relates to the use of a “controlled” vacuum source that may be used to aspirate saliva. However, these aspiration devices require a large amount of interaction between the device operator and the subject, and during collection of the sample it is necessary periodically to observe the device to determine when an adequate sample of saliva has been collected.

Many of the devices that fall within these categories have the disadvantage that the device must be further processed in a laboratory where trained personnel and/or specialised equipment are available to extract the sample from the device and/or analyse the sample. In addition, some procedures require cultures to be forwarded to laboratories for evaluation and thus delay further the commencement of appropriate treatment. Due to the serious nature of infections that may be caused by Strep A, it is important to diagnose its presence in an early stage of infection.

Another device which has been used to assay oral fluids is disclosed in U.S. Pat. No. 6,303,081 (Mink), wherein a hydrophobic capillary matrix is used to transport oral fluids to a lateral chromatography strip. However, such devices may not be suitable for collection of whole saliva from a plurality of regions of the mouth. Thus, the collection of saliva may be from a single gland. As a result, such collection dos not provide a sample that is representative of whole saliva and may not be reflective of the serum concentrations of certain analytes.

The aim of the present invention is to provide an improved device and method for the collection of oral fluids suitable for subsequent analysis.

### SUMMARY OF THE INVENTION

In a first aspect, the present invention provides a device for the collection of oral fluid from the oral cavity of a subject, the device comprising a sample collecting mouthpiece, the mouthpiece comprising a collecting chamber, introducible into the oral cavity of the subject, for collecting oral fluid with a resiliently deformable wall section around at least part of the collecting chamber, inward deformation of the resiliently deformable wall section causing compression of the collecting chamber so as to forcibly expel fluid from the chamber via an outlet; and a one-way valve which is in fluid communication with the sample receiving chamber outlet, wherein the one-way valve functions to allow fluid to be displaced from the device following compression of the sample collecting chamber but prevents ambient air flowing back through the valve into the device.

Preferably, the device of the present invention is introducible between the hard palate and tongue of the oral cavity of a subject.

In some embodiments of the invention, compression of the collecting chamber occurs between the hard palate and tongue of the oral cavity of the subject. Such compression may be caused by the subject applying pressure to the collecting chamber, causing deformation of the resiliently deformable wall section and subsequent compression of the collecting chamber, so as to forcibly expel fluid from the collecting chamber. In accordance with the present invention, the subject may apply pressure to the collecting chamber using part of the mouth, such as the tongue, teeth, jaw, etc.

In another embodiment, the collecting chamber is held between the teeth of the subject (e.g. the back teeth) and a “sipper tube” is placed over or under the tongue to collect the saliva as pressure applied by the jaw of the subject causes compression of the collecting chamber.

Conveniently, the device of the present invention does not require a power or vacuum source to facilitate collection of oral fluid.

Oral fluid must be able to enter the collecting chamber from the oral cavity. This may be provided for by the use of a porous material in the wall of the collecting chamber. Alternatively, and preferably, one or more sample holes are provided in the wall (comprised of non-porous material) of the collecting chamber, such that oral fluid may enter the collecting chamber through the sample holes. In a preferred embodiment, the one or more sample holes) are provided on the lower surface of the wall of the sample collecting chamber. If desired, a plurality of sample holes may be provided in a variety of locations around the wall of the collecting chamber.

The inventors have found that the sample holes may conveniently be generally circular, with a diameter of 0.5-5.0 mm, preferably about 2 mm.

Preferably the size and number of the sample holes is selected to reduce or minimise back-flow of oral fluid from the sample collecting chamber into the oral cavity of the subject, especially when the chamber is compressed. Conveniently, air may be drawn through the sample holes. Thus, the sample holes preferably have a dual function whereby firstly, the oral fluid from the oral cavity of a subject passes through the sample holes and enters the sample collecting chamber and secondly, the sample holes help to regulate the flow of oral fluid to ensure that there is minimal back-flow of oral fluid from the sample collecting chamber into the oral cavity.

By way of explanation, the collecting chamber will initially contain air at ambient pressure when it is introduced into the subject's mouth. Normally the subject will be encouraged to retain the collecting chamber in the mouth for a little while (e.g. one or two minutes), without compressing the chamber, to allow oral fluid to gather in the mouth. Then the collecting chamber is compressed. This expels the air from the chamber through the outlet and out of the device via the one-way valve, leaving a partial vacuum in the collecting chamber. Since the one-way valve does not permit the passage of air in the reverse direction (i.e. into the device), the partial vacuum draws in fluid from the oral cavity into the collecting chamber. Subsequent compression of the collecting chamber will expel the collected fluid through the outlet. The expelled fluid may be processed or manipulated in any desired manner.

For example, the fluid expelled from the collecting chamber may be passed through the one-way valve and immediately tested, or may be stored for testing later.

However, a single cycle of collection may not gather sufficient volume of fluid for testing purposes, and it may be desirable therefore to perform further collection cycles in order to collect a greater volume of fluid. In such circumstances, it is preferred that the device will comprise a sample receiving means or chamber. The sample receiving means should be large enough to hold a desired volume of oral fluid (e.g. 5 mls or 10 mls).

Typically the sample receiving means will be in fluid communication with the outlet of the sample collecting chamber. Preferably the sample receiving means will be in fluid communication with the one-way valve. Preferably the sample receiving means will be located intermediately between the outlet of the sample collecting chamber and the one-way valve.

Conveniently the sample receiving means comprises a tube, cylinder, stoppered container or the like. The collecting chamber is in fluid communication with the sample receiving means. Preferably, the fluid communication is provided by means of a delivery tube or conduit. Desirably the collecting chamber has at least one outlet which is joined to a proximal end region of the delivery tube or conduit, a distal end region of the delivery tube or conduit being in fluid communication with the sample receiving means.

Typically, the sample receiving means comprises a receiving chamber into which the fluid is collected, generally having a rigid wall. Preferably, the sample receiving means is substantially sealed by means of a lid, stopper or the like, in order to regulate the flow of air through the system. In a preferred embodiment, the delivery conduit is introduced into the sample receiving means through the lid. Conveniently, a hole or aperture may be provided on the lid of the sample receiving means, the hole having a diameter generally corresponding to the diameter of the delivery conduit, such that the delivery conduit may be inserted through the hole in a substantially air-tight manner, preventing the introduction of air into the system.

Advantageously, the sample receiving means is readily detachable from the device, so that fluid received in the device can be readily accessed for testing. For example, the sample receiving means may be retained in the device by a releasable retaining means, such as a screw-threaded engagement, a spring, clip, snap-fit or the like. Alternatively, the sample receiving means may be provided with a sampling port or the like, such that some or all of the fluid can be withdrawn e.g. by a pipette. Preferably the sampling port, if present, is sealable.

Preferably, the one-way valve is placed in fluid communication with the sample receiving means via a second conduit. In a preferred embodiment, a second hole is provided on the lid of the sample receiving means and the second conduit is inserted through the second hole in a substantially air tight manner, so as to prevent the introduction of air into the device.

The volume of oral fluid collected is determined, at least in part, by the volume of air displaced from the device, which in turn depends at least in part on the geometry of the collecting chamber. In order to facilitate the movement of oral fluid from the collecting chamber to the sample receiving means, the volume of air displaced by compression of the collecting chamber should preferably be greater than the volume of air in the delivery conduit.

In some embodiments, a mouthguard is provided towards one end of the sample collecting chamber. The provision of such a mouthguard aids the positioning of the device within the oral cavity of the subject. Conveniently the mouthguard is formed with an aperture, such that fluid expelled from the collecting chamber may pass through the aperture in the mouthguard and into a delivery conduit or sample receiving chamber.

In one embodiment, the device may incorporate an extendible mouthpiece, such that the sample collecting chamber may be placed in a desired region of the oral cavity of the subject.

Preferably, the wall of the sample collecting chamber comprises or consists of rubber latex, silicon latex or the like. In a specific embodiment, the sample collecting chamber includes a saliva-stimulating substance. Preferably, the saliva-stimulating substance is a natural substance. Alternatively, the saliva-stimulating substance may be an artificial substance. For example, the wall of the collecting chamber and/or a portion of the mouthguard (if present) may be coated, impregnated or otherwise treated with a saliva-stimulating substance. Suitable such substances may comprise artificial or natural sweeteners and/or acceptable weak acids (such as carboxylic acids) including but not limited to malic, ascorbic, tartaric and fumaric acids. In addition, to facilitate acceptance by young children, the device may be coated, impregnated or otherwise treated with a suitable flavouring (e.g. fruit flavour), especially one which is also saliva-stimulating.

The device may also comprise an anti-foaming agent, to reduce foaming of the saliva. The amount and type of anti-foaming agent would depend on the nature of the test to be performed, and would be selected so as not to interfere with the assay. The anti-foaming agent may conveniently be provided within the device prior to use, e.g. coated on an inside surface.

The device may also comprise an agent to reduce the viscosity of the oral fluid collected by the device. Reduced viscosity will improve the flow properties of the fluid, especially within narrow capillaries or porous materials. Such agents may, for example, be provided on the interior of part or all of the device e.g. as a coating, or deposited in bulk in the sample receiving and/or collecting chambers. A suitable agent is disclosed in U.S. Pat. No. 5,112,758.

As used herein, the term “oral fluid” refers to one or more fluids found in the oral cavity either individually or in combination. These include, but are not necessarily limited to, saliva and mucosal transudate. The oral fluids may comprise a combination of fluids from a number of sources (e.g. parotid, submandibular, sublingual, accessory glands, gingival mucosa and buccal mucosa).

The term “subject”, as used herein, refers to the test subject whose oral fluid is to be collected for testing. Preferably, the subject is a mammal. Most preferably, the subject is a human.

In some embodiments, the present invention provides a device for the collection of an oral fluid from a subject and a means for the detection and/or analysis of analytes present in the oral fluid. Conveniently, the device in accordance with the first aspect of the invention may comprise or be fluidically coupled to an apparatus for lateral flow chromatographic analysis of an oral fluid. The lateral flow chromatographic apparatus may, for example, be attached to the receiving means of the device such that when the oral fluid enters the receiving means, the oral fluid is delivered to an application zone of a lateral flow chromatographic strip, (preferably a lateral flow chromatographic immunoassay strip). In one embodiment, the oral fluid only passes from the receiving means to the assay device once a sufficient volume of fluid has been collected. Preferably, the lateral flow chromatographic apparatus is used to detect the presence of analytes in the oral fluid. More preferably, the apparatus is used to detect the presence of one or more antigens in the oral fluid. Most preferably, the apparatus is used to detect the presence of one or more Streptococcus A antigens in the oral fluid. Alternatively, the fluid collected using the device may be tested in a separate apparatus, assay device or other piece of equipment.

A “lateral flow chromatographic strip”, as used herein, refers to a test strip that is used for lateral flow chromatography, wherein a liquid test sample that is suspected of containing an analyte of interest is applied to an application zone of a lateral flow test strip. Preferably, the test strip is comprised of a porous matrix such as nitrocellulose, through which the test fluid and analyte suspended therein can move by capillary action from the application zone to a detection zone. The presence or absence of a visible signal at the detection zone reveals the presence or absence of the analyte of interest. A lateral flow chromatographic immunoassay strip is a lateral flow chromatographic strip which utilises at least one immunoglobulin or immunoglobulin-like molecule as a reagent.

As used herein, the term “analyte” is used to denote an unknown substance that is determined in a liquid medium.

Conveniently, the device of the present invention provides a system for rapid, safe, automatic, non-expensive and non-invasive collection of an oral fluid from a subject. The device of the present invention requires low sample manipulation and minimises the risk of sample contamination. The device of the present invention is particularly suitable for medical diagnostics. Devices in accordance with the present invention are therefore ideally suited for use in the home, office or work environment, or for point-of-care purposes and do not require laboratory facilities or the presence of trained medical personnel. In particular, the device is preferably suitable for self-use by the subject and may be used by adults or children. In one embodiment, the device may be reusable after washing. In another embodiment, the device may be disposable, either wholly or in part.

In a second aspect, the present invention provides a method of collecting an oral fluid from a subject, the method comprising the steps of introducing the collecting chamber of a device in accordance with the first aspect of the invention into the oral cavity of a subject, and retaining the device in the oral cavity until a sample of oral fluid has been collected.

In one embodiment in accordance with the second aspect of the invention, the method involves placing the sample collecting chamber between the hard palate of the oral cavity and the tongue of the subject. Preferably, the subject applies pressure to the sample collecting mouthpiece, causing compression of the sample collecting chamber. Such compression of the collecting chamber causes the oral fluid to be forcibly expelled from the sample collecting chamber. The collecting chamber may be retained in the oral cavity to allow a plurality of cycles in which the chamber is compressed briefly and then allowed to expand to its original size (or near original size), thereby to collect an adequate volume of oral fluid.

In another embodiment, the invention provides a method of performing a lateral flow chromatography assay to detect and/or analyse the presence of an analyte in an oral fluid sample collected by the method defined above. Preferably, the invention provides a method of detecting an antigen in the oral fluid. Most preferably, the invention provides a method of detecting the presence of a Streptococcus A antigen in the oral fluid.

In a third aspect, the invention provides a kit comprising apparatus for the collection of oral fluid from a subject in accordance with the first aspect of the invention and one or both of the following: a sample receiving means for placement, directly or indirectly, in fluid communication with the sample collecting chamber; and assay means for performing an assay on the sample of oral fluid collected by the device. In a particular embodiment, the kit may comprise apparatus for the lateral flow chromatographic analysis of an oral fluid and instructions for the use of the apparatus.

The invention will now be further described by way of illustrative example and with reference to the accompanying drawing,

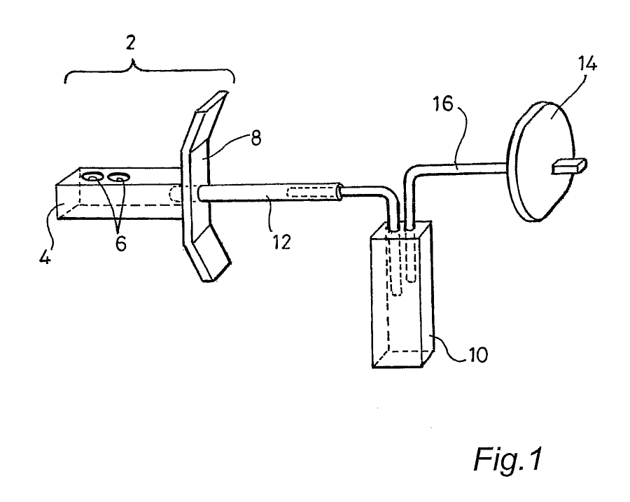

FIG. 1, which is a schematic representation of an embodiment of an assay device in accordance with the invention.

### EXAMPLE 1

With reference to FIG. 1, an embodiment of the device in accordance with the first aspect of the invention comprises a mouthpiece, indicated generally by reference numeral (2), comprising a sample collecting chamber (4) which is introducible into the oral cavity of a subject. The sample collecting chamber is typically 10-20 mm in length. The wall of the collecting chamber is made of rubber latex or silicon latex and is resiliently deformable. Sample holes (6) are provided in the wall of the sample collecting chamber, the sample holes having the dual function of allowing the passage of oral fluid from the oral cavity of the subject into the sample collection chamber (4), and minimising the back-flow of oral fluid from the sample collecting chamber into the oral cavity of the subject. The sample holes are approximately 2 mm in diameter. A mouthguard (8) is provided at the distal end of the mouthpiece (2).

The sample collecting chamber (4) has an outlet which is in fluid communication with a sample receiving means (10) via a delivery conduit (12). The delivery conduit (12) has an outer diameter of approximately 4 mm and an internal diameter of approximately 2 mm. Compression of the sample collecting chamber causes oral fluid to be forcibly expelled from the chamber into the sample receiving means. The sample receiving means (10) is substantially sealed by means of a lid and is in fluid communication with a one-way valve (14). Two holes are provided on the lid so as to allow the introduction of two conduits. One conduit (the delivery tube, 12) is in communication with the sample collecting chamber (2). A second conduit (16) is in fluid communication with the one-way valve (14).

The one-way valve (14) allows the displacement of air from the device following compression of the sample collecting chamber. However, the one-way valve (6) (Lee TKLA) does not allow the passage of air back through the device, thus tightly regulating the movement of air within the device.

The sample receiving means (10) is constructed from a 1.5 ml screw-top microcentrifuge tube (Starstedt™) and the tubing is comprised of PTFE (Cole-Palmer 20SW, 6417-31). The components of the device as described above are held in place by the use of a high melting point adhesive.

The device of the first aspect as described above may be used in accordance with the second aspect of the invention. In use, the sample collecting chamber (4) is introduced into the oral cavity of a subject. Oral fluid from the oral cavity is collected in the sample collecting chamber through sample holes (6) provided in the wall of the sample collecting chamber (4). The subject applies pressure to the sample collecting chamber (4), causing compression of the collecting chamber (4) so as to forcibly expel oral fluid from the sample collecting chamber initially into the delivery tube (12) thence into the sample receiving means (10).

Following compression, the subject releases the force exerted on the wall of the sample collecting chamber (4). Since the wall is resiliently deformable the sample collecting chamber (4) returns to its original size. This expansion results in a reduced pressure within the chamber, thus drawing in air and/or oral fluid, which can in turn be expelled from the chamber by subsequent compression. In this way, an adequate volume of oral fluid can be accumulated in the sample receiving means.

### EXAMPLE 2

The present inventors conducted a series of experiments with the aim of determining the optimal location of the sample holes (6) in the wall of the sample collecting chamber (4). In particular, investigations were carried out to determine whether it was preferable for the sample holes (6) to be provided in the upper or lower surface of the wall of the sample collecting chamber (4). Such tests were carried out with ten individual subjects. Each subject was instructed to test three devices having sample holes (6) provided on the upper surface of the wall of the chamber (4) and three devices having sample holes (6) provided on the lower surface of the wall of the chamber (4). The subjects were directed to apply pressure to the chamber (4) for 1 minute. At the end of each test the volume of oral fluid that had been collected was measured (in μl).

The results of these experiments are presented in Table 1 and demonstrate that samples of oral fluid from the individual subjects were efficiently collected using the device of the present invention. In addition, the results showed that the provision of sample holes (6) in the lower surface of the wall of the sample collecting chamber improved the performance of the device when compared with the performance observed when the sample holes (6) were provided in the upper surface of the wall of the chamber.

TABLE 1

| Donor | Upper | Upper | Upper | Lower | Lower | Lower |
|---|---|---|---|---|---|---|
| 1 | 400 | 600 | 700 | 1000 | 800 | 900 |
| 2 | 0 | 0 | 0 | 100 | 400 | 100 |
| 3 | 800 | 50 | 1000 | 500 | 1000 | 1000 |
| 4 | 700 | 1000 | 1000 | 800 | 1200 | 1000 |
| 5 | 600 | 400 | 1000 | 500 | 400 | 700 |
| 6 | 0 | 0 | 0 | 0 | 0 | 200 |
| 7 | 0 | 0 | 0 | 0 | 0 | 100 |
| 8 | 200 | 200 | 200 | 50 | 400 | 200 |
| 9 | 0 | 200 | 100 | 0 | 0 | 0 |
| 10 | 0 | 600 | 800 | 0 | 600 | 600 |

| | | |
|---|---|---|
| Average | 351.667 μl | 418.333 μl |
| Failure rate | 0.36667 | 0.26667 |
| Complete person failures | 0.3 | 0.1 |

1. A device for the collection of oral fluid from a subject's oral cavity, the device comprising;
a sample collecting mouthpiece, the mouthpiece comprising
a collecting chamber defined by a collecting chamber wall, introducible into the oral cavity of the subject, for collecting oral fluid; and
a resiliently deformable wall section of the collecting chamber wall, defining at least part of the collecting chamber such that compression of the resiliently deformable wall forcibly expels a collected fluid from the collecting chamber via a collecting chamber outlet; and
a one-way valve in fluid communication with the collecting chamber outlet, wherein the one-way valve allows fluid to be displaced from the device following compression of the resiliently deformable wall but prevents ambient air flowing back through the valve into the device.

2. A device according to claim 1, further comprising a sample receiving chamber for receiving oral fluid expelled from the collecting chamber.

3. A device according to claim 1, wherein the sample receiving chamber is in fluid communication with the collecting chamber and with the one-way valve.

4. A device according to claim 3, wherein the sample receiving chamber is located at an intermediate location along a fluid flow path between the collecting chamber and the one-way valve.

5. A device according to claim 2, wherein the sample receiving chamber is readily detachable from the device to allow access to oral fluid accumulated in the sample receiving chamber.

6. A device according to claim 1, wherein the resiliently deformable wall section comprises rubber latex or silicon latex.

7. A device according to claim 1, wherein the wall of the collecting chamber comprises a plurality of apertures or holes to allow ingress of oral fluid into the collecting chamber.

8. A device according to claim 7, wherein the plurality of holes are located on an underside or lower surface of the collecting chamber when the chamber is introduced in its normal position into the oral cavity.

9. A device according to claim 1, further comprising one or more of the following: a saliva-stimulating substance; an anti-foaming agent; and a saliva viscosity-reducing agent.

10. A device according to claim 1, which is wholly or partly disposable after a single use.

11. A device according to claim 1, further comprising an assay apparatus for performing an assay on oral fluid collected by the device.

12. A device according to claim 11, wherein the assay apparatus comprises a lateral flow chromatographic strip.

13. A device according to claim 11, wherein the assay apparatus is adapted for the detection of one or more Streptococcus A antigens.

14. (canceled)

15. A method of collecting an oral fluid from a subject, the method comprising the steps of:
introducing the collecting chamber of a device in accordance with claim 1 into the oral cavity of the subject, and
retaining the device in the oral cavity until a sample of oral fluid has been collected.

16. A kit for collection of oral fluid from a subject, the kit comprising a device in accordance with claim 1,
a sample receiving chamber for placement, directly or indirectly, in fluid communication with the collecting chamber of the device; and
an assay apparatus for performing an assay on the sample of oral fluid collected by the device.
